(19) United States
(12) Patent Application Publication
(10) Pub. No.: US 2008/0308112 A1
(43) Pub. Date: Dec. 18, 2008

(54) System and Method for Reducing Snoring and/or Sleep Apnea of Sleeping Person

(75) Inventor: Ronaldus Maria Aarts, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., Eindhoven (NL)

(21) Appl. No.: 12/097,762

(22) PCT Filed: Dec. 18, 2006
(86) PCT No.: PCT/IB06/54910

§ 371 Date: Jun. 17, 2008

(30) Foreign Application Priority Data
Dec. 20, 2005 (EP) 05112470.9

(51) Int. Cl. A61F 5/56

(52) U.S. Cl. 128/848

(57) ABSTRACT

System and method for reducing snoring and sleep apnea of a sleeping person (1), the system (2) comprising at least one sensor (4) for detecting occurrence and/or likeliness of occurrence of the snoring and/or sleep apnea and for producing a sensor signal (A) indicative of the occurrence and/or likeliness of the occurrence of the snoring and sleep apnea and a processor unit (6) for determining from the sensor signal whether the occurrence of the snoring and/or sleep apnea of the sleeping person is likely, wherein the system is provided with a stimulator (10) controllable by the processor unit, wherein the stimulator is arranged to trigger the sleeping person to change position using a triggering signal.

### FIELD OF THE INVENTION

The invention relate to a system and method for reducing snoring and/or sleep apnea of a sleeping person.

### BACKGROUND OF THE INVENTION

The term “apnea” means “no breath”. Such absence of breath occurs in sleep apnea, which is a serious, potentially life-threatening condition that is far more common than generally understood. Sleep apnea is a breathing disorder characterized by brief interruptions of breathing during sleep.

A system and method are known, for instance, from U.S. Pat. No. 6,454,724. This publication discloses an apnea monitor and alarm for monitoring the breathing of an individual. In response to an interruption in the cyclical rhythm of breathing an alarm is sounded, which awakens the individual.

### SUMMARY OF THE INVENTION

It is an object of the present invention to improve the above system and method.

According to the present invention, a system is provided, the system comprising at least one sensor, for instance an acoustical sensor, for detecting occurrence and/or likeliness of occurrence of the snoring and/or sleep apnea and for producing a sensor signal indicative of the occurrence and/or likeliness of the occurrence of the snoring and/or sleep apnea; a processor unit for determining from the sensor signal whether the occurrence of the snoring and/or sleep apnea of the sleeping person is likely; and a stimulator controllable by the processor unit, wherein the stimulator is arranged to trigger the sleeping person to change position using a triggering signal, while the person remains asleep.

A yet unrecognized problem of the system and method from U.S. Pat. No. 6,454,724 is that the awakening of the individual seriously interrupts a night's rest. One or more of such interruptions could cause an undesirable fatigue during for the individual during daytime.

Using the stimulator, the person does not need to be brought to an awakened state in order to allow normal breathing to be resumed. The stimulator may simply be arranged such, that the sleeping person changes position so that breathing will be resumed and/or snoring will be reduced. Suitably, the person's soft palate changes position for allowing that breathing will be resumed and/or snoring will be reduced while the person remains asleep.

In an advanced embodiment of the present invention, the at least one sensor is formed as an array of acoustical sensors, such as a microphone array. Such an array can be employed to separate acoustical sources, so that, in use, breathing sound of a bed partner can be separated from breathing sound of the user of the system.

Such a stimulator can, for example, be arranged to exert a mechanical force on the sleeping person to trigger the sleeping person to change position. The stimulator can also comprise an acoustical emitter, wherein the emitter is arranged to emit a sound having a suitable pitch, length and/or intensity for triggering the sleeping person to change position. Another possibility is that the stimulator is arranged to electrically trigger the sleeping person to change position. A stimulator for electrical triggering could, for instance, comprise at least one transcutaneous electrical nerve stimulation (TENS) device and/or at least one galvanic vestibular stimulation (GVS) coil.

Preferably, the system comprises a sleep detection apparatus for determining whether a person is asleep. When the system is provided with such a sleep detection apparatus, the system can be arranged to automatically cease detecting occurrence and/or likeliness of occurrence of the snoring and/or sleep apnea when the person is not asleep. Also, the processor unit may be arranged to start determining whether occurrence of the snoring and/or sleep apnea of the sleeping person is likely when the sleep detection apparatus has determined that the person has fallen asleep.

In an advanced embodiment, the sleep detection apparatus is arranged to determine the sleeping depth of the person. An advantage of such an embodiment is the possibility of arranging the processor unit to control the stimulator for triggering the sleeping person in accordance with the sleeping depth. Thus, at least one triggering parameter, which defines the triggering signal, is adjusted. The triggering performed by the stimulator may be reduced in intensity if the person is not sleeping deeply. Conversely, the triggering may be increased if the person is sleeping fairly deep in order to be able to induce the change of position in spite of the deep sleep. Thus, the advanced embodiment can improve the accuracy of the stimulator of the system.

A further embodiment may be that the processor unit is arranged to determine from the signal whether the sleeping person is still asleep or has accidentally been awoken due to the triggering of the person. Such an embodiment may be a learning system, wherein the processor unit may be arranged to adjust at least one triggering parameter, which defines the triggering signal, the processor being arranged to adjust the triggering parameters on the basis of information concerning earlier triggering. Such information may concern whether the person has been awoken as a result to at least one earlier triggering event and, for instance, the sleeping depth of the person at the moment just before the at least one triggering event.

The basic idea is to provide a system and method for reducing snoring and/or sleep apnea of a sleeping person without prejudicing the quality of the person's rest when sleeping. This idea follows from the yet unrecognized problem that awakening the person has the negative side effect on the amount of rest, which is desired in a night's sleep.

These and other aspects of the invention will be apparent from and elucidated with reference to the embodiments described hereinafter with reference to the accompanying drawings, in which like reference signs refer to similar parts.

### BRIEF DESCRIPTION OF THE DRAWINGS

In the drawings,

FIG. 1 is a side view of a sleeping person in a first position using a first embodiment of a system according to the present invention and a schematic view of the embodiment of the system;

FIG. 2 is a side view of the sleeping person in a second position, the person using the system of FIG. 1;

FIG. 3 is an enlarged side view of the head of the sleeping person in the first position;

FIG. 4 is a side view of a sleeping person using another embodiment of the system according to the present invention and a schematic view of the embodiment of the system; and

FIG. 5 is a schematic view of a person using a CPAP apparatus of a third embodiment of the system according to the present invention.

### DESCRIPTION OF PREFERRED EMBODIMENTS

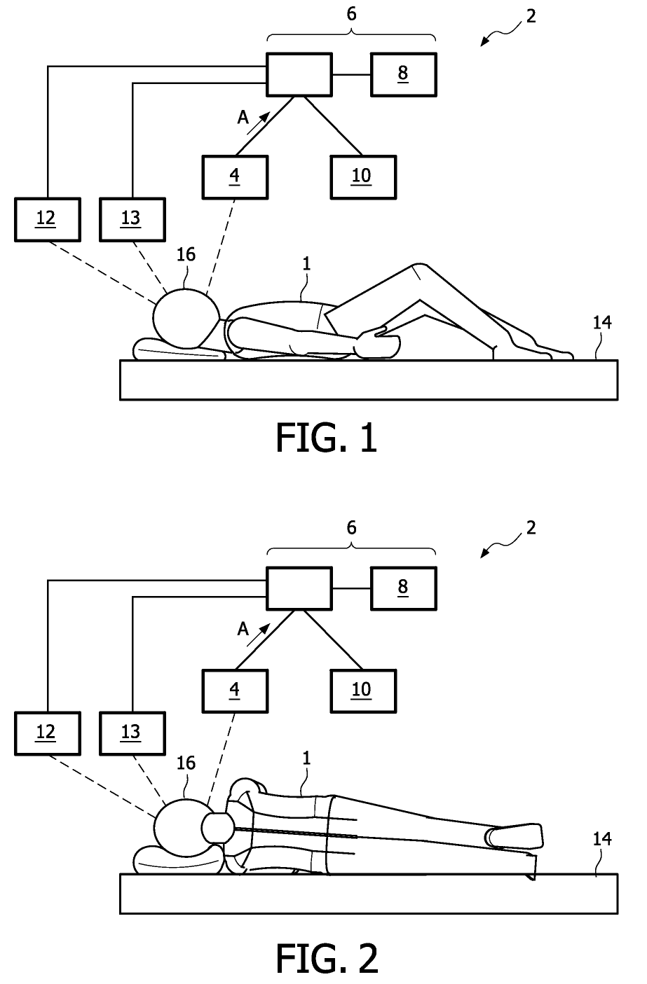

FIG. 1 shows a sleeping person 1 who is using a first embodiment of a system 2. The system 2 comprises a sensor 4, which is arranged to detect occurrence and/or likeliness of occurrence of the sleeping person's snoring and/or sleep apnea. The sensor 4 produces a signal A indicative of the occurrence and/or likeliness of the occurrence of the snoring and/or sleep apnea. For determining from the sensor signal A whether the occurrence of the snoring and/or sleep apnea of the sleeping person 1 is likely, the system 2 is provided with a processor unit 6 having a memory unit 8. Furthermore, the system 2 comprises a stimulator 10 arranged to trigger the sleeping person 1 to change position, while the person 1 remains asleep, using a triggering signal. The stimulator 10 may be an electrical stimulator, preferably a transcutaneous electrical nerve stimulation (TENS) device that is widely used in other applications. The electrical stimulator could also be a galvanic vestibular stimulation (GVS) coil or be a speaker arranged to generate a sound in order to trigger the sleeping person directly to change position. The speaker would preferably be arranged to emit a sound having a suitable pitch, length and/or intensity for triggering the sleeping person to change position. Alternatively, the stimulator 10 could indirectly trigger the sleeping person to change position. The stimulator 10 could, for instance, be a vibrator for vibrating a surface 14 on which the person 1 is sleeping, which would cause the sleeping person 1 to change position.

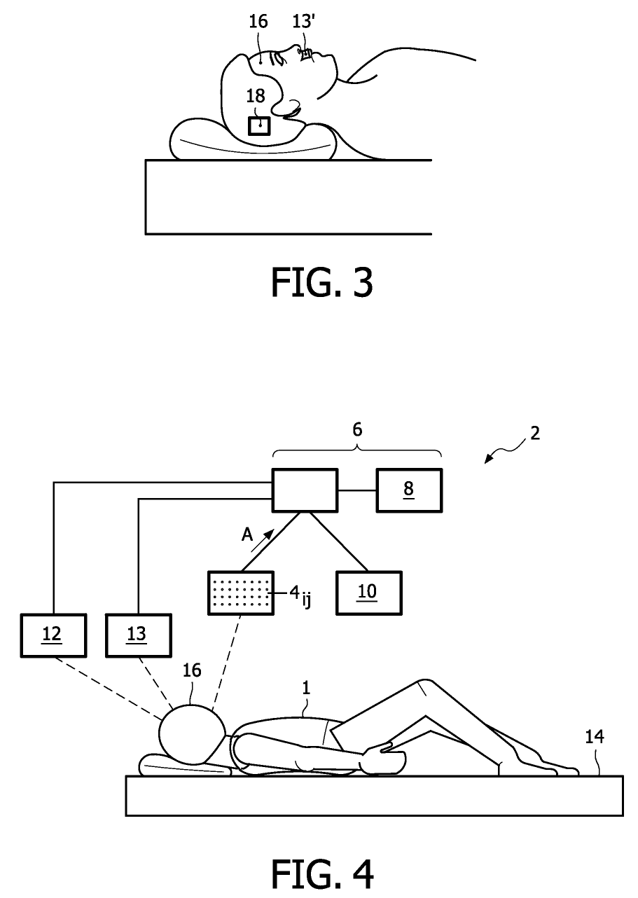

The processor unit 6 is arranged to control the stimulator 10. In addition, the system 2 may comprise a sleep detection apparatus 12 for determining whether a person is asleep and a polysomnography apparatus 13 for monitoring the sleeping person. Such a polysomnography apparatus 13 may be arranged to determine the sleeping depth of the sleeping person. The polysomnography apparatus 13 may also be arranged to monitor brain waves, eye movements, muscle activity, heartbeat and/or blood oxygen levels. For measuring respiration, the at least one sensor 4 may comprise a thermistor 13′ arranged to be placed between the sleeping person's nose and his upper lip (see FIG. 3). In use, the breath of the sleeping person influences a temperature and hence an electrical resistance of the thermistor 13′. The thermistor 13′ is preferably arranged to communicate wirelessly with the processor unit 6.

The sleep detection apparatus 12 and/or the polysomnography apparatus 13 may further be arranged to send data concerning the person's brain activity to the processor unit 6 which may be adapted, for instance, to derive from the brain activity a sleeping depth of the person 1. Alternatively, the polysomnography apparatus 13 could be arranged to derive from the brain activity a measure for sleeping depth of the person 1 and send this measure for sleeping to the processor unit 6.

Operation of the first embodiment of the system 2 will be explained referring to FIGS. 1 and 2.

In FIG. 1, the person 1 is sleeping on the surface 14. While the person 1 is asleep in a first position, the processor unit 6 uses sensor 4 to determine whether snoring and/or sleep apnea occurs and whether the snoring and/or sleep apnea is likely to occur. Also, the polysomnography apparatus 13 monitors the person during sleep. Data concerning the brain activity is sent to the processor unit 6, which determines from the brain activity the sleeping depth.

If snoring and/or sleep apnea occur or if the likeliness of occurrence thereof becomes high enough, the processor unit 6 controls the stimulator 10 to trigger the sleeping person to change position. In the first embodiment shown, the position change is such that breathing will be resumed and/or snoring will be reduced while the person remains asleep.

A manner in which breathing will be resumed and/or snoring will be reduced, is explained referring to FIG. 2. Generally, snoring and/or sleep apnea occurs, because the person's soft palate at least partially blocks his breathing airway (not shown in the Figures). By changing to a second position in which the person lies on a side the person's soft palate changes position, so that the blocking of his breathing airway is removed.

In order to prevent the person 1 from waking up as a result of a triggering of the stimulator 10, the triggering is adapted to the sleeping depth of the person 1 which depth is monitored using the polysomnography apparatus 13. To this end the memory 8 of the processor unit 6 is, in this embodiment of the system, provided with a table containing a triggering parameters, such as triggering power, as a function of the sleeping depth. However, if the person 1 nonetheless wakes as a result of the triggering performed by the stimulator 10, this is noted by the processor unit 6 from the sleep detection apparatus 12 and stored in the memory 8, thereby modifying the table. When the system 2 has been used long enough, the triggering of the stimulator 10 will very seldom wake the person 1, thereby improving the quality of a night's sleep.

If the person is awake, the sleep detection apparatus 12 will detect this and the system 2 will automatically cease detecting the occurrence of the snoring and/or sleep apnea and/or the likeliness of such occurrence. The processor unit 6 may also instruct the polysomnography apparatus 13 to cease monitoring the person, when the sleep detection apparatus 12 detects that the person is awake.

In FIG. 4, another more advanced, second embodiment of the system is shown. The second embodiment is quite similar to the first embodiment. However, in this embodiment, the at least one sensor is formed as an array of acoustical sensors, such as a microphone array 4ᵢⱼ as shown in FIG. 4. The array 4ᵢⱼ can be employed to separate acoustical sources. Separation of sources is a desirable feature in order to separate sound of the user of the system from sound emerging from other sources. For instance, breathing sound of a bed partner can be separated from breathing sound of the user. The array 4ᵢⱼ is, in FIG. 4, configured in the form of a matrix. However, the array 4ᵢⱼ may suitably be other configured in other ways.

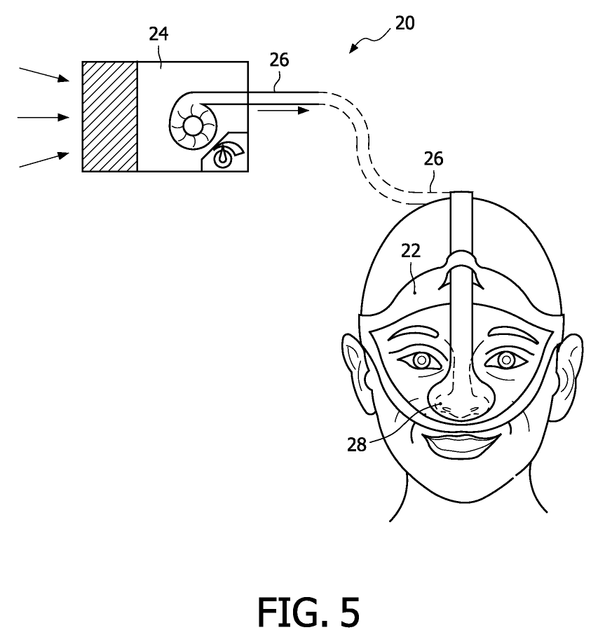

Referring to FIG. 5, a third embodiment of the system is explained. The third embodiment is also quite similar to the first embodiment. However, the third embodiment of the system 2 is provided with an apparatus 20 for relieving the sleeping person's airway. Preferably, the apparatus 20 is a continuous positive airway pressure (CPAP) apparatus (see FIG. 5), which comprises a nasal mask 22, a high flow fan 24 and a hose 26 for establishing a fluid connection for flow from the fan 24 to the mask 22. The apparatus 20 further comprises a nosepiece 18 for delivering air originating from the fan 24 to the nasal airway of the sleeping person 1. The CPAP apparatus 20 is controllable by the processor unit 6 which can be configured to activating the CPAP apparatus 20 if occurrence of the snoring and/or sleep apnea of the sleeping person are likely. However, because the CPAP apparatus 20 can be very effective in relieving the sleeping person's airway without the necessity of the sleeping person's changing position, it is also possible to apply this principle of activating the CPAP apparatus 20 if occurrence of the snoring and/or sleep apnea is likely, while omitting the stimulator 10.

In a modification, which can be applied in any embodiment of the system, the system 2 comprises an accelerometer 18 for determining acceleration. Preferably the accelerometer 18 is located at a position near the mouth and nose of the sleeping person 1. Such a position may be a position on the head 16 of the sleeping person 1 as shown in FIG. 3. The accelerometer 18 can measure acceleration occurring due to movement of the sleeping person. The accelerometer 18 can then send acceleration data to the processor unit 6, which is, in such a modification of the system 2, arranged to determine an estimation of a disturbance value in measurements performed by the sensor 4. Thus, the system 2 is able to compensate for the disturbance in sensor signal A resulting from movement of the sleeping person 1.

It should be stressed that the invention is not limited to the embodiments described above. It is possible to use any element suitable for the sleeping person to change position. Also, it may be possible to arrange the system to establish a link, for instance a wireless link, to a hospital and/or sleeping clinic, where relevant data concerning the person's sleep can be monitored, saved and studied. Furthermore, different kinds of sensors are applicable to detect occurrence and/or likeliness of occurrence of the snoring and/or sleep apnea. As already mentioned, acoustical sensors may be used. However, also an apparatus for measuring pulse transit time-derived respiratory parameters may be applied to detect the occurrence and/or likeliness of occurrence of snoring and/or sleep apnea without departing from the invention.

A sleeping surface provided with an actuator for prodding the back of the sleeping person in order for him to change position may be used as the stimulator in a system according to the present invention, as long as use of the stimulator will be able, at least generally, to operate while the person remains asleep. An effect is that the sleeping person is able to sleep continuously through the night. Therefore the system will significantly improve the quality of the person's sleep. Also, the sleep detection apparatus and the polysomnography apparatus can be integrated with each other.

It is noted that the word “comprising” does not exclude the presence other elements in an part of the apparatus or additional steps in a method. It is also noted that the word “a”/“an” does not exclude plurality. Furthermore, the reference signs in the claims are not to be construed as to limit the scope of the present invention. Furthermore, it is to be noted that the reference signs in the claims do not limit the scope of the invention.

1. System (2) for reducing snoring and/or sleep apnea of a sleeping person (1), the system (2) comprising
at least one sensor (4) for detecting occurrence and/or likeliness of occurrence of the snoring and/or sleep apnea and for producing a sensor signal (A) indicative of the occurrence and/or likeliness of the occurrence of the snoring and/or sleep apnea;
a processor unit (6) for determining from the sensor signal (A) whether the occurrence of the snoring and/or sleep apnea of the sleeping person (1) is likely; and
a stimulator (10) controllable by the processor unit (6), wherein the stimulator (10) is arranged to trigger the sleeping person to change position using a triggering signal, while the person (1) remains asleep.

2. System according to claim 1, wherein the processor unit is configured to control the stimulator (10) to trigger the sleeping person if occurrence of the snoring and/or sleep apnea of the sleeping person is likely.

3. System according to claim 1, wherein the stimulator (10) is arranged for the sleeping person (1) to change position in such a way that breathing will be resumed and/or snoring will be reduced while the person remains asleep.

4. System according to claim 3, wherein the change of position is such that the person's soft palate changes position for allowing that breathing will be resumed and/or snoring will be reduced while the person (1) remains asleep.

5. System according to claim 1, wherein the system comprises a sleep detection apparatus (12) for determining whether a person is asleep.

6. System according to claim 1, wherein the system comprises a polysomnography apparatus (13) for monitoring the sleeping person.

7. System according to claim 6, wherein the polysomnography apparatus (13) is arranged to determine the sleeping depth of the sleeping person.

8. System according to claims 7, wherein the processor unit (6) is arranged to control the stimulator (10) for triggering the sleeping person in accordance with the sleeping depth.

9. System according to claim 6, wherein the processor unit is arranged to start determining whether occurrence of the snoring and/or sleep apnea of the sleeping person is likely when the polysomnography apparatus (13) has determined that the person has fallen asleep.

10. System according to 6, wherein the processor unit and/or the sleep detection apparatus (12) is further arranged to determine from the signal whether the sleeping person (1) is still asleep or has been accidentally awoken due to the triggering of the person.

11. System according to claim 1, wherein the processor unit (6) is arranged to adjust at least one triggering parameter, which defines the triggering signal.

12. System according to claim 10, wherein the processor unit (6) is arranged to adjust the triggering parameters on the basis of information concerning earlier triggering.

13. System according to claim 1, wherein the stimulator (10) is arranged to exert a mechanical force on the sleeping person (1) to trigger the sleeping person to change position.

14. System according to claim 1, wherein the stimulator (10) comprises an acoustical emitter, wherein the emitter is arranged to emit a sound having a suitable pitch, length and/or intensity for triggering the sleeping person to change position.

15. System according to claim 1, wherein the stimulator (10) is arranged to electrically trigger the sleeping person to change position.

16. System according to claim 1, wherein the at least one sensor is an acoustical sensor.

17. System according to claim 16, wherein the at least one sensor comprises a sensor array (4ᵢⱼ).

18. System according to claim 17, wherein the processor unit (6) is arranged to separate acoustical sources.

19. Method for reducing snoring and/or sleep apnea, the method comprising the steps of:
producing a signal indicative of the occurrence and/or likeliness of occurrence of the snoring and/or sleep apnea using at least one sensor (4);
determining from the sensor signal whether occurrence of the snoring and/or sleep apnea of the sleeping person is likely; and
triggering the sleeping person to change position while the person remains asleep, if occurrence of the snoring and/or sleep apnea of the sleeping person is likely.

20. Method according to claim 19, wherein a sensor produces said signal, a processor determines from the sensor signal whether occurrence of the snoring and/or sleep apnea of the sleeping person is likely, and a stimulator (10) controllable by the processor unit (6) is arranged to trigger the sleeping person to change position using a triggering signal, while the person (1) remains asleep.
